(19) United States
(12) Patent Application Publication
(10) Pub. No.: US 2015/0232885 A1
(43) Pub. Date: Aug. 20, 2015

(54) PRODUCTION OF BIOKEROSENE WITH HYPERTHERMOPHILIC ORGANISMS

(71) Applicant: HYPERTHERMICS HOLDING AS, Hovdebygda (NO)

(72) Inventors: Michael Thomm, Regensburg (NO); Jan Remmereit, Volda (NO)

(21) Appl. No.: 14/421,493

(22) PCT Filed: Aug. 13, 2013
(86) PCT No.: PCT/IB2013/002891

§ 371 Date: Feb. 13, 2015

Related U.S. Application Data
(60) Provisional application No. 61/682,405, filed on Aug. 13, 2012.

(51) Int. Cl. C12P 5/02; C10G 1/00; C10L 1/04; C12P 3/00; C12P 7/54

(52) U.S. Cl. CPC C12P 5/02; C12P 3/00; C12P 7/54; C10L 1/04; C10G 1/002; C10G 1/00; C10L 2200/0469

(57) ABSTRACT

The present invention relates to processes from producing synthetic fuels from biolipid sources by treating the biolipids with biologically produced hydrogen gas, and the fuel stocks and fuels produced thereby.

### FIELD OF THE INVENTION

The present invention relates to processes from producing synthetic fuels from biolipid sources by treating the biolipids with biologically produced hydrogen gas, and the fuel stocks and fuels produced thereby.

### BACKGROUND OF THE INVENTION

The cost of conventional energy sources has increased dramatically in the last few years, and the use of many conventional energy sources such as oil, coal and nuclear power has been demonstrated to be harmful to the environment.

Many clean alternative energy sources have been developed or proposed. Such sources include solar energy, geothermal energy, wind energy, hydroelectric energy, hydrogen reactors and fuel cells. However, many of these sources are either expensive (solar energy) or limited by geographical concerns (geothermal, wind and hydropower).

Other alternative energy sources make use of biomass. However, those systems often involve the production of a secondary product such as ethanol or involve combusting the materials. These methods suffer from problems including contamination of the environment and requiring the use of valuable farmland to produce biomass.

Accordingly, what is needed in the art is alternative systems to utilize waste biomass materials or naturally available biomass materials to produce heat or electricity.

### SUMMARY OF THE INVENTION

The present invention relates to processes from producing synthetic fuels from biolipid sources by treating the biolipids with biologically produced hydrogen gas, and the fuel stocks and fuels produced thereby.

In some embodiments, the present invention provides processes comprising: treating a biomass with a culture of hyperthermophillic organisms to provide a biomass culture; culturing said biomass culture under conditions such that hydrogen gas is produced; and utilizing said hydrogen gas. In some embodiments, the biomass is an algal biomass. In some embodiments, the algal biomass is selected from the group consisting of a macroalgal biomass and a microalgal biomass. In some embodiments, the microalgal biomass comprises cyanobacteria. In some embodiments, the microalgal biomass comprises microalgae selected from the group consisting of Chlorella pyrenoidosa, Nannochloropsis salina, Porphyridium cruentum, Tetraselmis chuii, Chlamydomonas reinhardtii, Spirulina platensis, Schizotrychium sp. and Synechocystis sp. and combinations thereof. In some embodiments, utilizing said hydrogen gas comprises treating a biological lipid composition with said hydrogen gas to produce a hydrogenated lipid composition or straight chain paraffins. In some embodiments, the biological lipid composition comprises glycerides and/or free fatty acids from biological sources. In some embodiments, the biological sources are selected from the group consisting plant sources, algal sources, and combinations thereof. In some embodiments, the algal sources comprise microalgae. In some embodiments, the processes further comprise treating said straight chain paraffins with said hydrogen gas to produce synthetic paraffinic kerosene. In some embodiments, the processes further comprise the step of formulating fuel with said synthetic paraffinic kerosene. In some embodiments, the hyperthermophillic organism is selected from the group consisting of Pyrococcus, Thermococcus, Palaeococcus, Acidianus, Pyrobaculum, Pyrodictium, Pyrolobus, Methanopyrus, Methanothermus, Fervidobacterium and Thermotoga species, and combinations thereof. In some embodiments, culturing further produces acetate, and the processes further comprise the step of using said acetate as a feedstock for an algal biomass. In some embodiments, culturing further produces carbon dioxide, and the processes further comprise the step of using said carbon dioxide as a feedstock for an algal biomass. In some embodiments, heat from culture of said biomass is exchanged with an algal biomass. In some embodiments, the algal biomass is a residue after extraction of oil from said algal biomass. In some embodiments, culturing is at 80° C. or higher. In some embodiments, culturing is at about 80° C. to 110° C.

In some embodiments, the present invention provides straight chain paraffins produced by the processes described above.

In some embodiments, the present invention provides synthetic paraffinic kerosene produced by the processes described above.

In some embodiments, the present invention provides fuel produced by the processes described above.

### DESCRIPTION OF THE FIGURES

FIG. 1 provides a schematic depiction of system and process of the present invention.

### DEFINITIONS

As used herein, the term “biomass” refers to biological material which can be used as fuel or for industrial production. Most commonly, biomass refers to plant matter grown for use as biofuel, but it also includes plant or animal matter used for production of fibers, chemicals or heat. Biomass may also include biodegradable wastes that can be used as fuel. It is usually measured by dry weight. The term biomass is useful for plants, where some internal structures may not always be considered living tissue, such as the wood (secondary xylem) of a tree. This biomass became produced from plants that convert sunlight into plant material through photosynthesis. Sources of biomass energy lead to agricultural crop residues, energy plantations, and municipal and industrial wastes. The term “biomass,” as used herein, excludes components of traditional media used to culture microorganisms, such as purified starch, peptone, yeast extract but includes waste material obtained during industrial processes developed to produce purified starch. According to the invention, biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, algal biomasses, agricultural residues, municipal solid waste, industrial solid waste, sludge from paper manufacture, yard waste, wood and forestry waste. Examples of biomass include, but are not limited to, microalgae, macroalgae, corn grain, corn cobs, crop residues such as corn husks, corn stover, corn steep liquor, grasses, wheat, wheat straw, barley, barley straw, grain residue from barley degradation during brewing of beer, hay, rice straw, switchgrass, waste paper, sugar cane bagasse, sorghum, soy, components obtained from processing of grains, trees, branches, roots, leaves, wood chips, sawdust, shrubs and bushes, soybean hulls, vegetables, fruits, flowers and animal manure. In one embodiment, biomass that is useful for the invention includes biomass that has a relatively high carbohydrate value, is relatively dense, and/or is relatively easy to collect, transport, store and/or handle.

As used herein, the term “biomass by-products” refers to biomass materials that are produced from the processing of biomass.

As used herein, the term “bioreactor” refers to an enclosed or isolated system for containment of a microorganism and a biomass material. The “bioreactor” may preferably be configured for anaerobic growth of the microorganism.

As used herein, the term “hyperthermophilic organism” means an organism which grows optimally at temperatures above 80° C.

As used herein, the terms “degrade” and “degradation” refer to the process of reducing the complexity of a substrate, such as a biomass substrate, by a biochemical process, preferably facilitated by microorganisms (i.e., biological degradation). Degradation results in the formation of simpler compounds such as methane, ethanol, hydrogen, and other relatively simple organic compounds (i.e., degradation products) from complex compounds. The term “degradation” encompasses anaerobic and aerobic processes, including fermentation processes.

### DETAILED DESCRIPTION OF THE INVENTION

The present invention relates to the field of biomass degradation with hyperthermophilic organisms, and in particular to the use of hyperthermophilic degradation to produce heat, ethanol, hydrogen and other energy substrates from a biomass.

### A. Hyperthermophilic Organisms

The present invention contemplates the use of hyperthermophilic organisms for fermenting biomass. Thermophilic bacteria are organisms which are capable of growth at elevated temperatures. Unlike the mesophiles, which grow best at temperatures in the range of 25-40° C., or psychrophiles, which grow best at temperatures in the range of 15-20° C., thermophiles grow best at temperatures greater than 50° C. Indeed, some thermophiles grow best at 65-75° C., and hyperthermophiles grow at temperatures higher than 80° C. up to 113° C. (See e.g., J. G. Black, Microbiology Principles and Applications, 2d edition, Prentice Hall, New Jersey, [1993] p. 145-146; Dworkin, M., Falkow, S., Rosenberg, E, Schleifer, K-H., Stackebarndt E. (eds) The prokaryotes, third edition, volume 3, p. 3-28296 and p. 797-814 and p. 899-924; Madigan M., Martinko, J. Brock Biology of Microorganisms, eleventh edition, p. 430-441 and 414-415).

The thermophilic bacteria encompass a wide variety of genera and species. There are thermophilic representatives included within the phototrophic bacteria (i.e., the purple bacteria, green bacteria, and cyanobacteria), bacteria (i.e., Bacillus, Clostridium, Thiobacillus, Desulfotomaculum, Thermus, Lactic acid bacteria, Actinomycetes, Spirochetes, and numerous other genera). Many hyperthermophiles are archaea (i.e., Pyrococcus, Thermococcus, Thermotoga, Sulfolobus, and some methanogens). There are aerobic as well as anaerobic thermophilic organisms. Thus, the environments in which thermophiles may be isolated vary greatly, although all of these organisms are isolated from areas associated with high temperatures. Natural geothermal habitats have a worldwide distribution and are primarily associated with tectonically active zones where major movements of the earth's crust occur. Thermophilic bacteria have been isolated from all of the various geothermal habitats, including boiling springs with neutral pH ranges, sulfur-rich acidic springs, and deep-sea vents. In general, the organisms are optimally adapted to the temperatures at which they are living in these geothermal habitats (T. D. Brock, “Introduction: An overview of the thermophiles,” in T. D. Brock (ed.), Thermophiles: General, Molecular and Applied Microbiology, John Wiley & Sons, New York [1986], pp. 1-16; Madigan M., Martinko, J. Brock Biology of Microorganisms, eleventh edition, p. 442-446 and p. 299-328). Basic, as well as applied research on thermophiles has provided some insight into the physiology of these organisms, as well as promise for use of these organisms in industry and biotechnology.

The present invention is not limited to the use of any particular hyperthermophilic organism. In some embodiments, mixtures of hyperthermophilic organisms are utilized. In some embodiments, the hyperthermophiles are from the archaeal order Thermococcales, including but not limited to hyperthermophiles of the genera Pyrococcus, Thermococcus, and Palaeococcus. Examples of particular organisms within these genera include, but are not limited to, Pyrococcus furiosus, Thermococcus barophilus, T. aggregans, T. aegaeicus, T. litoralis, T. alcaliphilus, T. sibiricus, T. atlanticus, T. siculi, T. pacificus, T. waiotapuensis, T. zilligi, T. guaymasensis, T. fumicolans, T. gorgonarius, T. celer, T. barossii, T. hydrothermalis, T. acidaminovorans, T. profundus, T. stetteri, T. kodakaraenis, T. peptonophilis. In some embodiments, aerobic hyperthermophilic organisms such as Aeropyrum pernix, Sulfolobus solfataricus, Metallosphaera sedula, Sulfolobus tokadaii, Sulfolobus shibatae, Thermoplasma acidophilum and Thermoplasma volcanium are utilized. While in other embodiments, anaerobic or facultative aerobic organisms such as Pyrobaculum calidifontis and Pyrobaculum oguniense are utilized. Other useful archaeal organisms include, but are not limited to, Sulfolobus acidocaldarius and Acidianus ambivalens. In some embodiments, the hyperthermophilic organisms are bacteria, such as Thermus aquaticus, Thermus thermophilus, Thermus flavus, Thermus rubes; Bacillus caldotenax, Geobacillus stearothermophilus, Anaerocellum thermophilus, Thermoactinomyces vulgaris, and members of the order Thermotogales, including, but not limited to Thermotoga elfeii, Thermotoga hypogea, Thermotoga maritima, Thermotoga neapolitana, Thermotoga subterranean, Thermotoga thermarum, Petrotoga miotherma, Petrotoga mobilis, Thermosipho africanus, Thermosipho melanesiensis, Fervidobacterium islandicum, Fervidobacterium nodosum, Fervidobacterium pennavorans, Fervidobacterium gondwanense, Geotoga petraea, Geotoga subterranea. In some preferred embodiments, the microorganism preferably has the characteristics of Thermatoga strain MH-1, Accession No. DSM 22925 or Thermatoga strain MH-2, Accession No. DSM 22926. In some preferred embodiments, the microorganism preferably has the characteristics of at least one the Thermatoga strains SG1, RJ16, SG7, Pb12, S1, VL4-L8B, Pb19, VL4-L7A, Pb1, S3, S3-L1B, S3-L3A, PB10 LL 8B, RQ7, and LA10-L2B.

In some embodiments, hyperthermophilic strains of the above organisms suitable for fermenting biomass will be selected by screening and selecting for suitable strains. In still further embodiments, suitable strains will be genetically modified to include desirable metabolic enzymes, including, but not limited to hydrolytic enzymes, proteases, alcohol dehydrogenase, and pyruvate decarboxylase. See, e.g., (Bra/u, B., and H. Sahm [1986] Arch. Microbiol. 146:105-110; Bra/u, B. and H. Sahm [1986] Arch. Microbiol. 144:296-301; Conway, T., Y. A. Osman, J. I. Konnan, E. M. Hoffmann, and L. O. Ingram [1987] J. Bacteriol. 169:949-954; Conway, T., G. W. Sewell, Y. A. Osman, and L. O. Ingram [1987] J. Bacteriol. 169:2591-2597; Neale, A. D., R. K. Scopes, R. E. H. Wettenhall, and N. J. Hoogenraad [1987] Nucleic Acid. Res. 15:1753-1761; Ingram, L. O., and T. Conway [1988] Appl. Environ. Microbiol. 54:397-404; Ingram, L. O., T. Conway, D. P. Clark, G. W. Sewell, and J. F. Preston[1987] Appl. Environ. Microbiol. 53:2420-2425). In some embodiments, a PET operon is introduced into the hyperthermophile. See U.S. Pat. No. 5,000,000, incorporated herein by reference in its entirety.

### B. Biomass and Organic Matter

The present invention contemplates the degradation of biomass with hyperthermophilic organisms. The present invention is not limited to the use of any particular biomass or organic matter. Suitable biomass and organic matter includes, but is not limited to, microalgae, macroalgae, sewage, agricultural waste products, brewery grain by-products, food waste, organic industry waste, forestry waste, crops, grass, seaweed, plankton, algae, fish, fish waste, corn potato waste, sugar cane waste, sugar beet waste, straw, paper waste, chicken manure, cow manure, hog manure, switchgrass and combinations thereof. In some embodiments, the biomass is harvested particularly for use in hyperthermophilic degradation processes, while in other embodiments waste or by-products materials from a pre-existing industry are utilized.

In some preferred embodiments, the biomass is lignocellulosic. In some embodiments, the biomass is pretreated with cellulases or other enzymes to digest the cellulose. In some embodiments, the biomass is pretreated by heating in the presence of a mineral acid or base catalyst to completely or partially hydrolyze hemicellulose, de-crystallize cellulose, and remove lignin. This allows cellulose enzymes to access the cellulose.

In still other preferred embodiments, the biomass is supplemented with minerals, energy sources or other organic substances. Examples of minerals include, but are not limited, to those found in seawater such as NaCl, MgSO₄×7H₂O, MgCl₂×6H₂O, CaCl₂×2H₂O, KCl, NaBr, H₃BO₃, SrCl₂×6H₂O and KI and other minerals such as MnSO₄×H₂O, FeSO₄×7H₂O, CoSO₄×7H₂O, ZnSO₄×7H₂O, CuSO₄×5H₂O, KAl(SO₄)₂×12H₂O, Na₂MoO₄×2H₂O, (NH₄)₂Ni(SO₄)₂×6H₂O, Na₂WO₄×2H₂O and Na₂SeO₄.

Examples of energy sources and other substrates include, but are not limited to, purified sucrose, fructose, glucose, starch, peptone, yeast extract, amino acids, nucleotides, nucleosides, and other components commonly included in cell culture media.

In other embodiments, the biomass that is utilized has been previously fermented by another process. Surprisingly, it has been found that hyperthermophilic organisms are capable of growing on biomass that has been previously fermented by methanogenic microorganisms.

In some embodiments, biomass that contains or is suspected of containing human pathogens is treated by the hyperthermophilic process to destroy the pathogenic organisms. In some preferred embodiments, the biomass is heated to about 80° C. to 120° C., preferably to about 100° C. to 120° C., for a time period sufficient to render pathogens harmless. In this manner, waste such a human sewage may be treated so that it can be further processed to provide a safe fertilizer, soil amendment of fill material in addition to other uses.

In some preferred embodiments, the biomass is an algae, most preferably a marine algae (seaweed) or microalgae. In some embodiments, marine algae is added to another biomass material to stimulate hydrogen and/or acetate production. In some embodiments, the biomass substrate comprises a first biomass material that is not marine algae and marine algae in a concentration of about 0.01% to about 50%, weight/weight (w/w), preferably 0.1% to about 50% w/w, about 0.1% to about 20% w/w, about 0.1% to about 10% w/w, about 0.1% to about 5% w/w, or about preferably 1.0% to about 50% w/w, about 1.0% to about 20% w/w, about 1.0% to about 10% w/w, or about 1.0% to about 5% w/w. The present invention contemplates the use of a wide variety of seaweeds, including, but not limited to, algaes such as cyanobacteria (blue-green algae), green algae (division Chlorophyta), brown algae (Phaeophyceae, division Phaeophyta), and red algae (division Rhodophyta). In some embodiments, the microalgae is selected from Chlorella pyrenoidosa, Nannochloropsis salina, Porphyridium cruentum, Tetraselmis chuii, Chlamydomonas reinhardtii, Spirulina platensis, and Synechocystis sp. and combinations thereof. In some embodiments, the brown algae is a kelp, for example, a member of genus Laminaria (Laminaria sp.), such as Laminaria hyperborea, Laminaria digitata, Laminaria abyssalis, Laminaria agardhii, Laminaria angustata, Laminaria appressirhiza, Laminaria brasiliensis, Laminaria brongardiana, Laminaria bulbosa, Laminaria bullata, Laminaria complanata, Laminaria dentigera, Laminaria diabolica, Laminaria ephemera, Laminaria farlowii, Laminaria inclinatorhiza, Laminaria multiplicata, Laminaria ochroleuca, Laminaria pallid, Laminaria platymeris, Laminaria rodriguezii, Laminaria ruprechtii, Laminaria sachalinensis, Laminaria setchellii, Laminaria sinclairii, Laminaria solidugula and Laminaria yezoensis or a member of the genus Saccharina (Saccharina sp.), such as Saccharina angustata, Saccharina bongardiana, Saccharina cichorioides, Saccharina coriacea, Saccharina crassifolia, Saccharina dentigera, Saccharina groenlandica, Saccharina gurjanovae, Saccharina gyrate, Saccharina japonica, Saccharina kurilensis, Saccharina latissima, Saccharina longicruris, Saccharina longipedales, Saccharina longissima, Saccharina ochotensis, Saccharina religiosa, Saccharina sculpera, Saccharina sessilis, and Saccharina yendoana. In some embodiments, the brown algae if from one of the following following genera: Fucus, Sargassum, and Ectocarpus.

### C. Degradation and Energy Production

In preferred embodiments of the present invention, one or more populations of hyperthermophilic organisms are utilized to degrade biomass. In some embodiments, the biomass is transferred to a vessel such as a bioreactor and inoculated with one or more strains of hyperthermophilic organisms. In some embodiments, the environment of the vessel is maintained at a temperature, pressure, redox potential, and pH sufficient to allow the strain(s) to metabolize the feedstock. In some preferred embodiments, the environment has no added sulfur or inorganic sulfide salts or is treated to remove or neutralize such compounds. In other, embodiments, reducing agents, including sulfur containing compounds, are added to the initial culture so that the redox potential of the culture is lowered. In some preferred embodiments, the environment is maintained at a temperature above 45° C. In still further embodiments, the environment is maintained at between 55 and 90° C. In still further embodiments, the culture is maintained at from about 80° C. to about 110° C. depending on the hyperthermophilic organism utilized. In some preferred embodiments, sugars, starches, xylans, celluloses, oils, petroleums, bitumens, amino acids, long-chain fatty acids, proteins, or combinations thereof, are added to the biomass. In some embodiments, water is added to the biomass to form an at least a partially aqueous medium. In some embodiments, the aqueous medium has a dissolved oxygen gas concentration of between about 0.2 mg/liter and 2.8 mg/liter. In some embodiments, the environment is maintained at a pH of between approximately 4 and 10. In some embodiments, the environment is preconditioned with an inert gas selected from a group consisting of nitrogen, carbon dioxide, helium, neon, argon, krypton, xenon, and combinations thereof. While in other embodiments, oxygen is added to the environment to support aerobic degradation.

In other embodiments, the culture is maintained under anaerobic conditions. In some embodiments, the redox potential of the culture is maintained at from about −125 mV to −850 mV, and preferably below about −500 mV. Surprisingly, in some embodiments, the redox potential is maintained at a level so that when a biomass substrate containing oxygen is added to an anaerobic culture, any oxygen in the biomass is reduced thus removing the oxygen from the culture so that anaerobic conditions are maintained.

In some embodiments, where lignocellulosic materials are utilized, the cellulose is pre-treated as described above. The pre-treated cellulose is enzymatically hydrolyzed either prior to degradation in sequential saccharification and degradation or by adding the cellulose and hyperthermophile inoculum together for simultaneous saccharification and degradation.

It is contemplated that degradation of the biomass will both directly produce energy in the form of heat (i.e., the culture is exothermic or heat-generating) as well as produce products that can be used in subsequent processes, including the production of energy. In some embodiments, hydrogen, methane, and ethanol are produced by the degradation and utilized for energy production. In preferred embodiments, these products are removed from the vessel. It is contemplated that removal of these materials in the gas phase will be facilitated by the high temperature in the culture vessel. These products may be converted into energy by standard processes including combustion and/or formation of steam to drive steam turbines or generators. In some embodiments, the hydrogen is utilized in fuel cells. In some embodiments, proteins, acids and glycerol are formed which can be purified for other uses or, for example, used as animal feeds.

In some embodiments, the culture is maintained so as to maximize hydrogen production. In some embodiments, the culture is maintained under anaerobic conditions and the population of microorganisms is maintained in the stationary phase. Stationary phase conditions represent a growth state in which, after the logarithmic growth phase, the rate of cell division and the one of cell death are in equilibrium, thus a constant concentration of microorganisms is maintained in the vessel.

In some embodiments, the degradation products are removed from the vessel. It is contemplated that the high temperatures at which the degradation can be conducted facilitate removal of valuable degradation products from the vessel in the gas phase. In some embodiments, methane, hydrogen and/or ethanol are removed from the vessel. In some embodiments, these materials are moved from the vessel via a system of pipes so that the product can be used to generate power or electricity. For example, in some embodiments, methane or ethanol are used in a combustion unit to generate power or electricity. In some embodiments, steam power is generated via a steam turbine or generator. In some embodiments, the products are packages for use. For example, the ethanol, methane or hydrogen can be packaged in tanks or tankers and transported to a site remote from the fermenting vessel. In other embodiments, the products are fed into a pipeline system.

In other preferred embodiments, the present invention provides a process in which biomass is treated in two or more stages with hyperthermophilic organisms. In some embodiments, the process comprise a first stage where a first hyperthermophilic organism is used to treat a biomass substrate, and a second stage where a second hyperthermophilic organism is used to treat the material produced from the first stage. Additional hyperthermophilic degradation stages can be included. In some embodiments, the first stage utilizes Pyroccoccus furiosus, while the second stage utilizes Thermotoga maritima. A preferred embodiment is depicted in FIG. 4. In some preferred embodiments, the material produced from the second stage, including acetate, is further utilized as a substrate for methane production as described in more detail below.

In some embodiments, H₂ and/or CO₂ produced during hyperthermophilic degradation of a biomass are combined with methane from a biogas facility to provide a combustible gas. In some embodiments, H₂ and/or CO₂ producing during hyperthermophilic degradation of a biomass are added to a biogas reactor to increase production of methane.

The present invention also provides systems, compositions and processes for degrading biomass under improved conditions. In some embodiments, a hyperthermophile strain derived from a marine hyperthermophile is utilized and the biomass is provided in a liquid medium that comprises less than about 0.2% NaCl. In some embodiments, the NaCl concentration ranges from about 0.05% to about 0.2%, preferably about 0.1% to about 0.2%. In some embodiments, the preferred strain is MH-2 (Accession No. DSM 22926). In these embodiments, the biomass is suspended in a liquid medium so that it can be pumped into a bioreactor system. It is contemplated that the lower salt concentration allows use of the residue left after degradation for a wider variety of uses and also results in less corrosion of equipment. Furthermore, the lower salt concentration allows for direct introduction of the degraded biomass containing acetate, or liquid medium containing acetate that is derived from the hyperthermophilic degradation, into a biogas reactor.

In further embodiments, the processes and microorganisms described herein facilitate degradation of biomass using concentrations of hyperthermophilic organisms that have not been previously described. In some embodiments, the concentration of the hyperthermophilic organism in the bioreactor is greater than about 10⁹ cells/ml. In some embodiments, the cell concentration ranges from about 10⁹ cells/ml to about 10¹¹ cells/ml, preferably from about 10⁹ cells/ml to about 10¹⁰ cells/ml.

In still further embodiments, the present invention provides processes that substantially decrease the hydraulic retention time of a given amount of biomass in a reactor. Hydraulic retention time is a measure of the average length of time that a soluble compound, in this case biomass suspended or mixed in a liquid medium, remains in a constructed reactor and is presented in hours or days. In some embodiments, the hydraulic retention time of biomass material input into a bioreactor in a process of the present invention is less than about 10 hours, preferably less than about 5 hours, more preferably less than about 4 hours, and most preferably less than about 3 or 2 hours. In some embodiments, the hydraulic retention time in a hyperthermophilic degradation process of the present invention is from about 1 to about 10 hours, preferably from about 1 to 5 hours, and most preferably from about 2 to 4 hours.

### D. Production of Biofuels

In some embodiments, the present invention provides systems and processes for producing biokerosene and/or biodiesel. In some preferred systems and processes, processes and systems for degradation of a biomass substrate, such as an algal biomass substrate, by hyperthermophilic organisms is synergistically combined with systems and processes for biolipid production, for example from cultivation of suitable microalgaes, macroalgaes, plants, or combinations thereof. In some preferred embodiments, H₂ produced by the thermophilic or hyperthermophilic degradation of a biomass is reacted with a lipid composition, preferably a biologic lipid composition, to produce a substrate or intermediate, such as a hydrogenated lipid composition or straight chain paraffins, that can be used as a biofuel or in the production of a biofuel such as biokerosene or biodiesel. It is contemplated that the use of H₂ produced by biological processes allows the production of biofuels that are essentially 100% renewable.

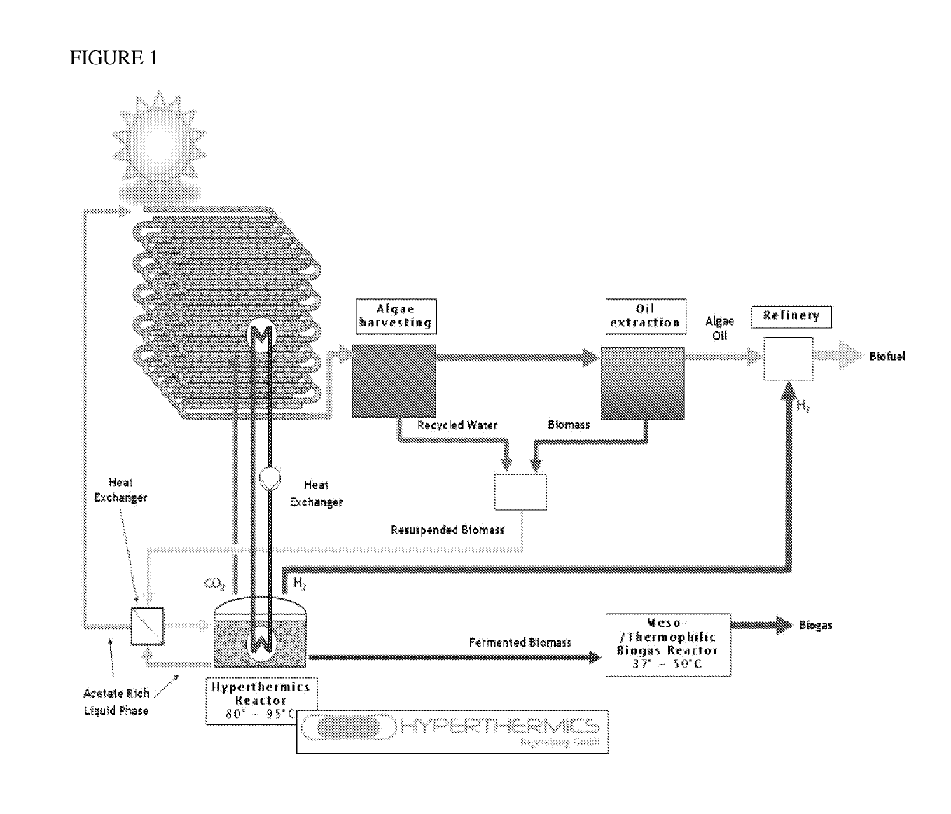

An exemplary embodiment is depicted in FIG. 1. As depicted in FIG. 1, the systems and processes of the present invention provide for the cultivation of algae, for example microalgae. The algae are preferably provided with sunlight, nutrients, and carbon dioxide (for example, emitted carbon dioxide). The algae are allowed to grow (i.e., the algal biomass increases) and then harvested for oil extraction.

Any suitable species of algae or prokaryotic cyanobacteria may be used in the present invention. In preferred embodiments, the algae is a microalgae, for example, a diatom (Bacillariophyceae), green algae (Chlorophyceae), or golden algae (Chrysophyceae). The algae may preferably grow in fresh or saline water. In some preferred embodiments, microalgae from one or more of the following genera are utilized: Oscillatoria, Chlorococcum, Synechococcus, Amphora, Nannochloris, Chlorella, Nitzschia, Oocystis, Ankistrodesmus, Isochrysis, Dunaliella, Botryococcus, Spirulina, Synechocystis, Tetraselmis, Chlamydomonas, Porphyridium, and Chaetocerus. In some embodiments, the microalgal biomass comprises microalgae selected from the group consisting of Chlorella pyrenoidosa, Nannochloropsis salina, Porphyridium cruentum, Tetraselmis chuii, Chlamydomonas reinhardtii, Spirulina platensis, and Synechocystis sp. and combinations thereof.

In certain embodiments, the algae may be genetically engineered to contain one or more isolated nucleic acid sequences that enhance oil production or provide other characteristics of use for algal culture, growth, harvesting or use.

In various embodiments, algae may be separated from the medium and various algal components, such as oil, may be extracted using any method known in the art. For example, algae may be partially separated from the medium using a standing whirlpool circulation, harvesting vortex and/or sipper tubes. Alternatively, industrial scale commercial centrifuges or tricanters of large volume capacity may be used to supplement or in place of other separation methods. Such centrifuges may be obtained from known commercial sources (e.g., Cimbria Sket or IBG Monforts, Germany; Alfa Laval A/S, Denmark). Centrifugation, sedimentation and/or filtering may also be of use to purify oil from other algal components. Separation of algae from the aqueous medium may be facilitated by addition of flocculants, such as clay (e.g., particle size less than 2 microns), aluminum sulfate or polyacrylamide. In the presence of flocculants, algae may be separated by simple gravitational settling, or may be more easily separated by centrifugation. Flocculent-based separation of algae is disclosed, for example, in U.S. Patent Appl. Publ. No. 20020079270, incorporated herein by reference.

The skilled artisan will realize that any method known in the art for separating cells, such as algae, from liquid medium may be utilized. For example, U.S. Patent Appl. Publ. No. 20040121447 and U.S. Pat. No. 6,524,486, each incorporated herein by reference, disclose a tangential flow filter device and apparatus for partially separating algae from an aqueous medium. Other methods for algal separation from medium have been disclosed in U.S. Pat. Nos. 5,910,254 and 6,524,486, each incorporated herein by reference. Other published methods for algal separation and/or extraction may also be used. (See, e.g., Rose et al., Water Science and Technology 1992, 25:319-327; Smith et al., Northwest Science, 1968, 42:165-171; Moulton et al., Hydrobiologia 1990, 204/205:401-408; Borowitzka et al., Bulletin of Marine Science, 1990, 47:244-252; Honeycutt, Biotechnology and Bioengineering Symp. 1983, 13:567-575).

In various embodiments, algae may be disrupted to facilitate separation of oil and other components. Any method known for cell disruption may be utilized, such as ultrasonication, French press, osmotic shock, mechanical shear force, cold press, thermal shock, rotor-stator disruptors, valve-type processors, fixed geometry processors, nitrogen decompression or any other known method. High capacity commercial cell disruptors may be purchased from known sources. (E.g., GEA Niro Inc., Columbia, Md.; Constant Systems Ltd., Daventry, England; Microfluidics, Newton, Mass.) Methods for rupturing microalgae in aqueous suspension are disclosed, for example, in U.S. Pat. No. 6,000,551, incorporated herein by reference.

The algae may be cultured in a variety of systems. In some embodiments, the algae are cultured in fresh water or saline open ponds. In other embodiments, the algae are cultured in closed bioreactor systems such as fiber optic filaments, polymer tubing, and polymer bags.

As further depicted in FIG. 1, the systems and processes further comprise a hyperthermophilic bioreactor for degradation of a biomass by hyperthermophilic organisms as described in detail above. In some embodiments, the algal biomass residue resulting from the oil extraction is used as the biomass substrate in the hyperthermophilic bioreactor.

In some embodiments, acetate, CO₂ and/or other degradation products produced by fermentation with hyperthermophilic organisms are used for the culture of algae. In these embodiments, the degradation products (e.g., acetate), preferably contained in liquid fermentation broth, is introduced into a culture system for the production of algae. In some embodiments, the liquid fermentation broth from the hyperthermophilic bioreactor contains acetate and is delivered to the algae culture system. Preferably, the bioreactor and culture system are in fluid communication, but in alternative embodiments, the acetate-containing substrate may be delivered via tanker or other means.

As further depicted, in some embodiment the systems further comprise a heat exchanger so heat can be exchanged between the hyperthermophilic bioreactor and the algal cultivation system.

In some embodiments, the biomass resulting from the hyperthermophilic culture is processed in a mesophilic biogas reactor resulting in the production of methane. One of the main products of fermentation with the hyperthermophilic organisms is acetate. The present invention provides novel processes for utilizing acetate to produce energy.

In some embodiments, acetate produced by fermentation with hyperthermophilic organisms is used for the production of methane or biogas. In these embodiments, the acetate, preferably contained in liquid fermentation broth, is introduced into a bioreactor containing methanogenic microorganisms. Examples of methanogens that are useful in bioreactors of the present invention include, but are not limited to, Methanosaeta sp. and Methanosarcina sp. The methane produced by this process can subsequently be used to produce electricity or heat by known methods.

The use of a wide variety of bioreactors, also known as biodigesters, is contemplated. Examples include, but are not limited to, floating drum digesters, fixed dome digesters, Deenbandhu digesters, bag digesters, plug flow digesters, anaerobic filters, upflow anaerobic sludge blankets, and pit storage digesters. Full-scale plants that are suitable for use in the present invention can be purchased from providers such as Schmack A G, Schwandorf, D E. These systems may be modified to accept introduction acetate from the hyperthermophilic bioreactors of the present invention. In some preferred embodiments, the methanogen bioreactor is in fluid communication with the hyperthermophilic bioreactor. In some embodiments, the liquid fermentation broth from the hyperthermophilic bioreactor contains acetate and is delivered to the methanogen bioreactor. Preferably, the bioreactors are in fluid communication, but in alternative embodiments, the acetate-containing substrate may be delivered via tanker or other means.

In some embodiments, biomass is input into a bioreactor containing hyperthermophilic microorganisms. The biomass is preferably provided in a liquid medium. In some embodiments, the biomass has been previously degraded by microorganisms (e.g., the biomass may be the residue from a biogas reactor as depicted), biomass that has not been previously degraded or fermented by a biological process, or a mixture of the two. Degradation products from the hyperthermophilic bioreactor include H₂ and acetate. In some embodiments, acetate from the hyperthermophilic reactor is introduced into the biogas reactor. In some embodiments, the acetate is at least partially separated from the biomass residue in the hyperthermophilic reactor. In some embodiments, an aqueous solution comprising the acetate is introduced into the biogas reactor. In other embodiments, a slurry comprising the biomass residue and acetate is introduced into the biogas reactor. In some embodiments, the aqueous solution or slurry are pumped from the hyperthermophilic reactor into the biogas reactor. As described above, in some preferred embodiments, the aqueous solution or slurry have a NaCl concentration of less than about 0.2%. In some embodiments, H₂ is removed from the system, while in other embodiments, H₂ and other products including CO₂, are introduced into the biogas reactor. In some embodiments, the systems include a heat transfer system, such as the Organic Rankine Cycle. It is contemplated that production of acetate by degradation of biomass with hyperthermophilic microorganisms either before or after biogas production an increase the efficiency of use of a biomass material as compared to known biogas processes.

In some embodiments, hydrogen gas (H₂) produced from the hyperthermophilic degradation of biomass is used to treat biolipid compositions (e.g., algal or plant oils) to produce hydrogenated lipids and/or straight chain paraffins, which are a feedstock for the production of synthetic paraffinic kerosene and biodiesel. In some preferred embodiments, the Fischer-Tropsch (FT) process is utilized.

The FT process produces a broad spectrum of linear, paraffinic hydrocarbons. These can be converted using conventional refining techniques into a range of products including diesel, gasoline, kerosene and chemicals. In some embodiments, the hydrogen gas (H₂) produced from the hyperthermophilic degradation of biomass is further used to treat straight chain paraffins in a cracking process to produce synthetic paraffinic kerosene (SNP) which comprises small, highly branched hydrocarbon molecules. In some preferred embodiments, the SNP is used to formulate fuels, for example, jet fuel or diesel fuel.

In some embodiments, the plant/algal oils are used to make biodiesel. In some of these embodiments, H₂ produced by hyperthermophilic fermentation is used to hydrogenate plant or algal oils to produce a hydrogenated lipid composition. The hydrogenated lipid composition is then used to make biodiesel. See, for example, the Neste Oil process. A variety of other methods for conversion of photosynthetic derived materials into biodiesel are known in the art and any such known method may be used in the practice of the instant invention. For example, the algae may be harvested, separated from the liquid medium, lysed and the oil content separated. The algal-produced oil will be rich in triglycerides. Such oils may be converted into biodiesel using well-known methods, such as the Connemann process (see, e.g., U.S. Pat. No. 5,354,878, incorporated herein by reference). Standard transesterification processes involve an alkaline catalyzed transesterification reaction between the triglyceride and an alcohol, typically methanol. The fatty acids of the triglyceride are transferred to methanol, producing alkyl esters (biodiesel) and releasing glycerol. The glycerol is removed and may be used for other purposes.

Preferred embodiments may involve the use of the Connemann process (U.S. Pat. No. 5,354,878). In contrast to batch reaction methods (e.g., J. Am. Oil Soc. 61:343, 1984), the Connemann process utilizes continuous flow of the reaction mixture through reactor columns, in which the flow rate is lower than the sinking rate of glycerine. This results in the continuous separation of glycerine from the biodiesel. The reaction mixture may be processed through further reactor columns to complete the transesterification process. Residual methanol, glycerine, free fatty acids and catalyst may be removed by aqueous extraction. The Connemann process is well-established for production of biodiesel from plant sources such as rapeseed oil and as of 2003 was used in Germany for production of about 1 million tons of biodiesel per year (Bockey, “Biodiesel production and marketing in Germany.”)

### EXPERIMENTAL

### Example 1

### Use of Microalgae as Substrate for Hyperthermophilic Degradation

Thermotoga MH-1 (T. MH-1) was inoculated in 20 ml MM-I medium, supplemented with 0.05% yeast extract and with different amounts (1%-5% TS) of microalgae as substrate. The media were prepared by applying the microalgae in 120 ml serum bottles and adding 20 ml of MM-I medium (supplemented with 0.05% yeast extract) inside an anaerobic chamber. Hydrogen and acetate production were used as growth indicators, as determination of the cell density in microalgal media was not possible due to the high concentration of microalgal cells.

| | | | | Acetate | Acetate |
|---|---|---|---|---|---|
| | | max. H₂ yield | max. H₂ yield | yield per kg | yield per |
| | Substrate | per kg | per liter liquid | substrate | liter liquid |
| | characteristics | substrate (TS) | culture | (TS) | culture |
| Microalgae | | | | | |
| applied as | | | | | |
| substrate | | | | | |
| Chlorella | dry pellets | 31 l × kg⁻¹ | 306 ml × l⁻¹ | 46 g × kg⁻¹ | 456 mg × l⁻¹ |
| pyrenoidosa | | (on 1% TS) | (on 1% TS) | (on 1% TS) | (on 1% TS) |
| Nannochloropsis | freeze dried | 22 l × kg⁻¹ | 559 ml × l⁻¹ | 27 g × kg⁻¹ | 678 mg × l⁻¹ |
| salina | powder | (on 1% TS) | (on 5% TS) | (on 1% TS) | (on 5% TS) |
| Porphyridium | frozen cell | 28 l × kg⁻¹ | 459 ml × l⁻¹ | 35 g × kg⁻¹ | 618 mg × l⁻¹ |
| cruentum | paste | (on 1% TS) | (on 4% TS) | (on 1% TS) | (on 4% TS) |
| Tetraselmis chuii | frozen cell | 46 l × kg⁻¹ | 1547 ml × l⁻¹ | 50 g × kg⁻¹ | 1829 mg × l⁻¹ |
| | paste | (on 2% TS) | (on 4% TS) | (on 2% TS) | (on 4% TS) |
| Chlamydomonas | freeze dried | 11 l × kg⁻¹ | 106 ml × l⁻¹ | 14 g × kg⁻¹ | 141 mg × l⁻¹ |
| reinhardtii | powder | (on 1% TS) | (on 1% TS) | (on 1% TS) | (on 1% TS) |
| Cyanobacteria | | | | | |
| applied as | | | | | |
| substrate | | | | | |
| Spirulina | spray dried | 22 l × kg⁻¹ | 224 ml × l⁻¹ | 29 g × kg⁻¹ | 377 mg × l⁻¹ |
| platensis | powder | (on 1% TS) | (on 1% TS) | (on 1% TS) | (on 4% TS) |
| Synechocystis | freeze dried | 34 l × kg⁻¹ | 508 ml × l⁻¹ | 48 g × kg⁻¹ | 937 mg × l⁻¹ |
| spec. | powder | (on 1% TS) | (on 2% TS) | (on 1% TS) | (on 5% TS) |

### Example 2

### 10 Liter Fermentation with Thermotoga MH-1 on 5% Synechocystis Spec. As Substrate

The small scale experiment above demonstrates that T. MH-1 can grow on Synechocystis spec. cell powder. Now we examined the hydrogen and acetate yields in a 10 liter fermentation.

T. MH-1 was inoculated in 10 liter MM-I medium with 5% TS Synechocystis spec. cell powder as substrate and 0.05% TS yeast extract as additive. When the fermentation was finished, the fermentation broth was centrifuged to separate the solid phase from the liquid phase. The solid as well as the liquid phase were sent to Tormod Briseid (Bioforsk; Ås, Norway) to analyze the biogas potential of the HT-fermented microalgae.

Results:

On 5% Synechocystis spec. as substrate, T. MH-1 produced 907 ml/l hydrogen and 1.7 g/l acetate. This corresponds to 18 liter hydrogen and 34 g acetate per kg substrate. These yields are quite low, compared to other substrates, e.g. macroalgae waste (60-70 liter H2/kg; 70-120 g acetate/kg). We observed two hydrogen production phases in the course of the fermentation. The first H2-production phase peaked on February at 11:00 am, the second at 2:00 am. These two production phases refer to diauxie. Diauxie means that there are two different kinds of substrates available and T. MH-1 consumes them successively. When the first (the preferred) substrate is consumed, the bacteria have to change their metabolism before they can start to consume the second substrate.

### Example 3

### Small Scale Experiments with Thermotoga MH-1 on Extraction Residues of Spirulina and Schizochytrium from Biodiesel Production

In this experiment two microalgae (Spirulina platensis [powder/chips] and Schizochytrium sp. [powder]) which had previously undergone an oil extraction procedure for biodiesel production were used as substrate. The former is a prokaryote (cyanobacterium), the latter an eukaryote. The extraction residues were provided by the German company IGV GmbH in Potsdam (Brandenburg). Spirulina is the most widely produced microalgae worldwide with 3000 tons (dry weight) cultivated each year. They are a source of phycocyanin (a pigment for the food and beverage industry) and also used in human and animal nutrition as well as in the cosmetic industry. It produces also hydrogen under anaerobic conditions (2 μmol H₂*d⁻¹*mg cell dry weight⁻¹) in the presence and absence of light at 32° C.). Schizochytrium is a marine microalgal used for the production of the omega-3 fatty acid docosahexaenoic acid [DHA] (production amount: 25 tons cell dry weight in 2003). Thermotoga MH-1 was inoculated in 20 ml MM-I medium, supplemented with 0.05% yeast extract and with different amounts (1%-5% TS) of substrates. The media were prepared by applying the substrate in 120 ml serum bottles and adding 20 ml of MM-I medium (supplemented with 0.05% yeast extract) inside an anaerobic chamber. The media were inoculated with Thermotoga MH-1 without previous sterilization by autoclaving. Hydrogen and acetate production were used as growth indicators, as determination of the cell density was not possible due to the high concentration of microalgal biomass in the medium.

Thermotoga MH-1 grew on Spirulina platensis (chips) concentrations of 1-5% and the hydrogen production increased with increasing substrate concentration, suggesting that even higher hydrogen yields can be achievable. The highest amount of hydrogen was 22.9 mM (at 5% substrate conc.) which is almost equal to the one measured when starch was used as substrate (24.7 mM [control experiment]). The acetate production in this case (12.1 mM) was almost twice as high as the one found in the control experiment (6.9 mM).

| | substrate concentration | | | | | | |
|---|---|---|---|---|---|---|---|
| | 0% | 1% | 2% | 3% | 4% | 5% | |
| | S. platensis | S. platensis | S. platensis | S. platensis | S. platensis | S. platensis | 0.5% |
| | (chips) | (chips) | (chips) | (chips) | (chips) | (chips) | starch |
| Hydrogen | 2.2 mM | 8.0 mM | 15.0 mM | 17.4 mM | 20.6 mM | 22.9 mM | 24.7 mM |
| production | | | | | | | |
| Acetate | 0.4 mM | 3.7 mM | 6.7 mM | 9.1 mM | 9.8 mM | 12.1 mM | 6.9 mM |
| production | | | | | | | |

Thermotoga MH-1 grew on Spirulina platensis (powder) concentrations of 1-5%. The hydrogen production reached a maximum (13.0 mM) at a substrate concentration of 2% and decreased afterwards. Acetate concentration was highest (7.1 mM) at a substrate concentration of 3%. The highest hydrogen production was only half of that produced in the control experiment (i.e. when starch was used as substrate). The highest acetate production was equal to that of the control experiment.

| | substrate concentration | | | | | | |
|---|---|---|---|---|---|---|---|
| | 0% | 1% | 2% | 3% | 4% | 5% | |
| | S. platensis | S. platensis | S. platensis | S. platensis | S. platensis | S. platensis | 0.5% |
| | (powder) | (powder) | (powder) | (powder) | (powder) | (powder) | starch |
| Hydrogen | 2.2 mM | 9.2 mM | 13.0 mM | 12.9 mM | 4.1 mM | 2.0 mM | 24.7 mM |
| production | | | | | | | |
| Acetate | 0.4 mM | 3.3 mM | 4.9 mM | 7.1 mM | 3.8 mM | 3.7 mM | 6.9 mM |
| production | | | | | | | |

Thermotoga MH-1 grew on Schizochytrium sp. (powder) concentrations of 1-4%. Chips were not provided by the company in this case. The highest hydrogen production was reached by a substrate concentration of 1%. The hydrogen concentration decreased first slowly and then sharply by a substrate concentration of 4%. At a substrate concentration of 5% no hydrogen production could be determined. The highest hydrogen production (18.8 mM) corresponded to ¾ of the amount found in the control experiment. The highest acetate production (8.7 mM) was 25% higher with regard to the control.

| | substrate concentration | | | | | | |
|---|---|---|---|---|---|---|---|
| | 0% | 1% | 2% | 3% | 4% | 5% | |
| | Schizochytrium | Schizochytrium | Schizochytrium | Schizochytrium | Schizochytrium | Schizochytrium | |
| | sp. | sp. | sp. | sp. | sp. | sp. | 0.5% |
| | (powder) | (powder) | (powder) | (powder) | (powder) | (powder) | starch |
| Hydrogen | 2.2 mM | 18.8 mM | 15.7 mM | 13.6 mM | 1.9 mM | — | 24.7 mM |
| production | | | | | | | |
| Acetate | 0.4 mM | 8.7 mM | 7.5 mM | 7.2 mM | 2.4 mM | 1.9 mM | 6.9 mM |
| production | | | | | | | |

### Example 4

### Comparison of Hydrogen Production on S. platensis Biomass (Suitable for Human Consumption) and on Extraction Residues after Biodiesel Production

This example describes the use of Spirulina platensis residues following biodiesel production as a biomass substrate for hyperthermophilic degradation.

Results: Extraction residues from Spirulina platensis (chips) in a 5% concentration yielded the second highest hydrogen concentration (22.9 mM) measured in small scale hitherto. The highest hydrogen concentration was 25.0 mM when using 5% of Nannochloropsis salina (an eukaryote) as substrate. The third highest hydrogen concentration (22.7 mM) was reached with 5% Synechocystis sp. (which is—like Spirulina—a prokaryote). Extraction residues from Spirulina platensis (powder) yielded similar results as those reached with S. platensis biomass suitable for human consumption. A reasonable comparison between both is not possible since they were produced for totally different aims, come from different providers and have therefore probably been processed in quite different ways.

| | Substrate concentrate | | | | | | | |
|---|---|---|---|---|---|---|---|---|
| | no | 1% | 2% | 3% | 4% | 5% | 6% | 0.5% |
| | substrate | S. platensis | S. platensis | S. platensis | S. platensis | S. platensis | S. platensis | starch |
| H₂ production from | 2.0 mM | 10.0 mM | 9.2 mM | not studied | 8.9 mM | not studied | no growth | 20.6 mM |
| S. platensis biomass | | | | | | | | |
| [dried cells suitable for | | | | | | | | |
| human consumption] | | | | | | | | |
| H₂ production from | 2.2 mM | 9.2 mM | 13.0 mM | 12.9 mM | 4.1 mM | 2.0 mM | not studied | 24.7 mM |
| S. platensis (powder) | | | | | | | | |
| [residues after | | | | | | | | |
| biodiesel production] | | | | | | | | |
| H₂ production from | | 8.0 mM | 15.0 mM | 17.4 mM | 20.6 mM | 22.9 mM | not studied | |
| S. platensis (chips) | | | | | | | | |
| [residues after | | | | | | | | |
| biodiesel production] | | | | | | | | |

### Example 5

### 10 Liter Fermentation with Thermotoga MH-1 on 5% Nannochloropsis Sauna as Substrate

Thermotoga MH-1 was inoculated in 10 liter MM-I medium, supplemented with 0.05% yeast extract and with 5% (TS) of the microalga Nannochloropsis salina as substrate. Cell densities during the fermentation could not be determined due to the high concentration of microalgal cells. When the fermentation was finished, the fermentation broth was centrifuged to separate the solid phase from the liquid phase. The solid as well as the liquid phase were sent to Tormod Briseid (Bioforsk; Ås, Norway) to analyze the biogas potential of the HT-fermented microalgae.

T. MH-1 produced this time 751 ml/l hydrogen and 1720 mg/l acetate or 15 liter hydrogen and 34 g acetate per kg substrate. This represents an increase of 8% in hydrogen production and of 73% with regard to acetate when compared with a previous experiment where N₂-flushing before reduction was omitted by mistake. When Synechocystis sp. was used as substrate, a higher hydrogen yield was reached (18 l/kg) than with N. salina (15 l/kg) but that former fermentation lasted also almost twice as long. N. salina is metabolized more rapidly by T. MH-1 than Synechocystis and shows no diauxie). After 40 h of fermentation (with N. salina as substrate), a hydrogen concentration of 32 mM had already been reached and the metabolization of the substrate was completed. In contrast, when Synechocystis was used as substrate, the hydrogen concentration measured after 34 h was only ˜14 mM and the first metabolic phase was not yet finished.

| Organism | Thermotoga MH-1 | | Thermotoga MH-1 | | Thermotoga MH-1 | |
|---|---|---|---|---|---|---|
| Medium | MM-I | | MM-I | | MM-I | |
| Substrate TS | 5.0% Nannochloropsis salina | | 5.0% Nannochloropsis salina | | 5.0% Synechocystis spec. | |
| Additives | 0.05% yeast extract | | 0.05% yeast extract | | 0.05% yeast extract | |
| Fermentation time | 34 | h | 33.75 | h | 62.75 | h |
| (without lag-phase) | | | | | | |
| Hydrogen | | | | | | |
| Total amount | 695 ml/l | (31 mM) | 751 ml/l | (32 mM) | 907 ml/l | (40 mM) |
| Amount per kg substrate TS | 14 | liter/kg | 15 | liter/kg | 18 | liter/kg |
| Acetate | | | | | | |
| Total amount | 992 mg/l | (17 mM) | 1720 mg/l | (29 mM) | 1710 mg/l | (28 mM) |
| Amount per kg substrate TS | 20 | g/kg | 34 | g/kg | 34 | g/kg |
| Ethanol | | | | | | |
| Total amount | 82 mg/l | (1.8 mM) | 86 mg/l | (1.9 mM) | 74 mg/l | (1.6 mM) |
| Amount per kg substrate TS | 1.6 | g/kg | 1.7 | g/kg | l.5 | g/kg |
| Lactate | | | | | | |
| Total amount | 51 mg/l | (0.6 mM) | 33 mg/l | (0.4 mM) | 85 mg/l | (0.9 mM) |
| Amount per kg substrate TS | 1.0 | g/kg | 0.7 | g/kg | 17 | g/kg |

### Example 6

### Small Scale Experiments with Thermotoga MH-1 on Eukaryotic Microalgae Tetraselmis chuii

Tetraselmis chuii is a green algae used for aquaculture. The cell covering of these cells, the theca, has a crystalline substructure and is composed of hydroxyproline-rich glycoproteins associated with various polysaccharides. It does not contain cellulose (1).

Growth of Thermotoga MH-1 was examined on the eukaryotic microalgae Tetraselmis chuii. The substrate was provided as a frozen paste (concentrated by centrifugation) by the company BlueBioTech (bluebiotech.de/com/index.html)

Experimental Approach:

Thermotoga MH-1 was inoculated in 20 ml MM-I medium, supplemented with 0.05% yeast extract and with different amounts (1%-5% TS) of microalgae as substrate. The media were prepared by applying the microalgae in 120 ml serum bottles and adding 20 ml of MM-I medium (supplemented with 0.05% yeast extract) inside an anaerobic chamber. The serum bottles were afterwards inoculated without previous autoclaving.

Almost all hydrogen concentrations measured in this experiment are higher than 25 mM and those ones are highlighted red in the table below. The hydrogen concentration declined 17% (57 mM) in relation to the highest value when a substrate concentration of 5% was used.

| | Substrate concentration (TS) | | | |
|---|---|---|---|---|
| | No substrate | 1% T. chuii | 2% T. chuii | 3% T. chuii |
| Hydrogen | 54 ml/l (2.2 mM) | 429 ml/l (17.5 mM) | 1005 ml/l (41.1 mM) | 1071 ml/l (43.8 mM) |
| concentration | | | | |
| Acetate | 54. mg/l (0.9 mM) | 458 mg/l (7.6 mM) | 1003 mg/l (16.7 mM) | 1139 mg/l (19.0 mM) |
| concentration | | | | |

| | Substrate concentration (TS) | | |
|---|---|---|---|
| | 4% T. chuii | 5% T. chuii | 0.5% Starch |
| Hydrogen | 1690 ml/l (69.0 mM) | 1405 ml/l (57.4 mM) | 606 ml/l (24.7 mM) |
| concentration | | | |
| Acetate | 1829 mg/l (30.5 mM) | 1451 mg/l (24.2 mM) | 415 mg/l (6.9 mM) |
| concentration | | | |

The H₂ concentration measured in the small scale experiment with T. chuii is also slightly higher than the highest concentration measured in small scale with macroalgae (66 mM with 5% TS Laminaria hyperborea). This makes T. chuii one of the most promising substrates for hydrogen production in a HT process.

| | Substrate | | | |
|---|---|---|---|---|
| | | | | Saccharina latissima |
| | Palmaria palmata | Laminaria hyperborea | Saccharina latissima | waste |
| Hydrogen concentration | 31 mM on 5% TS | 66 Mm on 5% TS | 31 mM on 4% TS | Not tested |
| in small scale | | | | |
| Total hydrogen production | 130 mM on 5% TS | not carried out due to | 56 mM on 2% TS | 239 mM |
| in bioreactor | (10 \| reactor) | lack of substrate | (100 \| reactor) | (10 \| reactor) |

### Example 7

### 10 Liter Fermentation with Thermotoga MH-1 on 5% (TS) Laminaria saccharina Waste

T. MH-1 was cultivated in 10 liter MM-I medium, supplemented with 0.05% (w/v) yeast extract and with 500 g dried L. saccharina waste as substrate. The algae waste was purchased from an algae farm on the island of Sylt. According to Prof. Lüning, the carrier of the algae farm, the algae material is qualitatively comparable to floating refuse on the coasts of the Baltic and North Sea.

Results:

T. MH-1 grew to a cell density of 6.1×10⁹ cells/ml and reached a maximum H₂ production rate of 387 ml×h⁻¹×1⁻¹. This is the highest cell number and the highest H₂ production rate ever measured in our laboratory. The fermentation yielded 65 liter H₂ per kg substrate, which is not as high as for corn silage (116 liter per kg). But compared to a biogas fermentation with algae residues as substrate, the HT fermentation can yield much more energy rich products. The following table compares the HT results with results from biogas fermentations of algae from floating refuse.

TABLE 1
Comparison of a HT fermentation and biogas fermentation with algae refuse as
substrate. The red colored values are calculated under the assumption that the acetate,
produced in the HT process, can completely be converted to CH₄.

| | HT fermentation | Biogas fermentation¹⁾ |
|---|---|---|
| Substrate | algae refuse from an algae farm | algae from floating refuse |
| H₂ yield per kg substrate | 65 NI | — |
| Acetate yield per kg substrate | 110 g | — |
| | (could be converted to 41 \| CH₄) | |
| CH₄ yield per kg substrate | — | 6-34 \| |
| Energy from H₂ | 231 Wh - kg⁻¹ | |
| Energy from CH4 | — | 67-377 Wh kg⁻¹ |
| Theoretical energy yield by | 454 Wh kg⁻¹ | — |
| conversion of acetate to CH4 | | |
| and subsequent combustion | | |
| Total energy yield | 231-685 Wh kg⁻¹ | 67-377 Wh kg⁻¹ |

¹⁾see “Energie aus Tang un Algen?” http://vuet.dk/filer/slutkonf/energie_DE.pdf

### Example 8

### 10 Liter Fermentation with Thermotoga MH-1 on 10% (TS) Saccharina Latissima Waste

For industrial application high substrate concentrations are desirable in order to reduce the size (and the costs) of the fermentation facility. With this experiment we examined growth and productivity of T. MH-1 on 10% dried algae waste in a 10 liter fermenter. The dry matter content of fresh algae is usually about 10-20%, so the H₂O content is about 80-90%. This means that 10% dry matter (as applied in this experiment) correspond to 50-100% fresh matter.

T. MH-1 was inoculated in 10 liter MM-I medium, supplemented with 0.05% (w/v) yeast extract and with 1 kg dried S. latissima waste as substrate. The fermenter was inoculated in the morning with a relatively high cell concentration of 5×10⁵ cells/ml.

Results:

After inoculation in the morning (7:45 am) with a pre-culture grown on starch, there was no indication for growth the whole day. T. MH-1 had a lag phase of about 10 hours, before growth on Saccharina latissima started. When N₂-stripping was started the next morning, T. MH-1 had already reached a cell density of 1.2×10¹⁰ cells/ml. This outstanding high cell density, which was never obtained before, was reached without active H₂ removal (N₂-stripping). Only the gas outlet of the fermenter was opened in order to allow gas emanation. On basis of our previous results and what is known from literature, we assumed that N₂-stripping was essential to avoid growth inhibition by H₂-accumulation. But our results show that this is not true for T. MH-1 grown on Saccharina latissima waste. In previous experiments there was also another kind of inhibition, caused by a still unknown substance, which prohibited growth of T. MH-I beyond 5×10⁹ cells/ml. This inhibition was not observed on S. latissima waste and so T. MH-1 could reach a cell density of 2×10¹⁰ cells/ml, which is by far the highest we ever measured.

The maximum hydrogen production rate of 502 ml×min⁻¹×1⁻¹ was also the highest ever measured and was obtained without N₂-stripping. This rate is almost 5-times higher compared to corn silage (103 ml×min⁻¹ 1⁻¹).

| | Duration of experiment | | | | | |
|---|---|---|---|---|---|---|
| | 47.75 hours | | | | | |
| | (10 hours lag phase) | | 24.75 hours | | 41.5 hours | |
| Substrates | 10% (TS) S. lastissima waste | | 5% (TS) S. latissima waste | | 1.05% (TS) corn silage | |
| Additives | 0.5% (w/v) yeast extract | | 0.5% (w/v) yeast extract | | 0.5% (w/v) yeast extract | |
| Generation time | n.d.¹⁾ | | 40 | min | 1.8 | hours |
| Maximum cell density | 2.1 × 10¹⁰ cells/ml | | 6.1 × 10⁹ cells/ml | | 4.5 × 10⁹ cells/ml | |
| Hydrogen | | | | | | |
| Duration of H₂ measurement | 34 | hours | 20 | hours | 25.5 | hours |
| Total amount produced | 5360 ml/l | (239 mM) | 3263 ml/l | (146 mM) | 1213 ml/(54 mM) | |
| Maximum production rate | 502 ml × l⁻¹ × h⁻¹³⁾ | | 387 ml × l⁻¹ × h⁻¹ | | 103 ml × l⁻¹ 1 × h⁻¹ | |
| H₂ per kg TS | 54 | liter/kg | 65 | liter/kg | 116 | liter/kg |
| Acetate | | | | | | |
| Total amount | 8238 mg/l | (137 mM) | 5493 mg/l | (91 mM) | 1831 mg/l | (30 mM) |
| Amount per kg TS | 82 | g/kg | 110 | g/kg | 174 | g/kg |
| Lactate | | | | | | |
| Total amount | 1961 mg/l | (21.8 mM) | 672 mg/l | (7.5 mM) | 430 mg/l | (4.8 mM) |
| Amount per kg TS | 20 | g/kg | 13 | g/kg | 41 | g/kg²⁾ |

### Example 9

### Screening of Various Thermatoga Strains on S. platensis Biomass

Additional Thermatoga strains were screened for hydrogen production using S. platensis as a substrate. The results are presented in the table below.

| Strain | H2 produced | Acetate produced | pH |
|---|---|---|---|
| SG1 | 39.43153525 | 18.157 | 5 |
| RJ16 | 36.93336803 | 17.354 | 5 |
| SG7 | 34.22795417 | 17.431 | 5 |
| Pb12 | 30.88499041 | 12.608 | 5 |
| S1 | 30.16742163 | 14.454 | 5.25 |
| VL4-L8B | 29.89990651 | 13.652 | 5 |
| Pb19 | 29.77807605 | 12.616 | 5 |
| VL4-L7A | 28.98839956 | 13.48 | 5.25 |
| Pb1 | 27.68024732 | 13.466 | 5.5 |
| S3 | 26.39138078 | 12.924 | 5.5 |
| S3-L1B | 23.70413935 | 12.457 | 5.5 |
| S3-L3A | 23.25316584 | 12.57 | 5.25 |
| PB10 LL 8B | 23.15988445 | 12.739 | 5.5 |
| RQ7 | 20.93481715 | 12.219 | 6 |
| LA10-L2B | 20.80919764 | 10.358 | 5.75 |
| MH.1 | 18.85552241 | 10.229 | 5.75 |
| VL20-L1A | 18.30486443 | 9.12 | 5.75 |
| NS-E | 16.67359371 | 9.204 | 6 |
| LA-4 | 16.11685021 | 7.03 | 6 |
| VL7-L5A | 15.25602872 | 9.437 | 6 |
| NE7 | 14.05150694 | 7.03 | 6 |
| VL4-L2A | 13.07682202 | 7.481 | 6 |
| VL20-L3B | 12.34475262 | 7.08 | 6 |
| VL7-L2B | 11.87064044 | 6.572 | 6.25 |
| Pb10 | 8.020921151 | 4.669 | 6.5 |
| Kol6TT-L3B | 7.482318504 | 3.84 | 6.5 |
| T. maritima | 4.67946147 | 3.121 | 6.5 |
| Kol5-L2B | 0 | 0.494 | 7 |
| AV18 | 0 | 0.59 | 7 |

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A process comprising:
treating a biomass with a culture of hyperthermophillic organisms to provide a biomass culture;
culturing said biomass culture under conditions such that hydrogen gas is produced;
utilizing said hydrogen gas.

2. The process of claim 1, wherein said biomass is an algal biomass.

3. The process of claim 2, wherein said algal biomass is selected from the group consisting of a macroalgal biomass and a microalgal biomass.

4. The process of claim 3, wherein said microalgal biomass comprises cyanobacteria.

5. The process of claim 3, wherein said microalgal biomass comprises microalgae selected from the group consisting of Chlorella pyrenoidosa, Nannochloropsis salina, Porphyridium cruentum, Tetraselmis chuii, Chlamydomonas reinhardtii, Spirulina platensis, Schizotrychium sp. and Synechocystis sp. and combinations thereof.

6. The process of claim 5, wherein said utilizing said hydrogen gas comprises treating a biological lipid composition with said hydrogen gas to produce a composition selected from the group consisting of straight chain paraffins and hydrogenated lipids.

7. The process of claim 6, wherein said biological lipid composition comprises glycerides and/or free fatty acids from biological sources.

8. The process of claim 7, wherein said biological sources are selected from the group consisting plant sources, algal sources, and combinations thereof.

9. The process of claim 8, wherein said algal sources comprise microalgae.

10. The process of claim 6, further comprising treating said straight chain paraffins with said hydrogen gas to produce synthetic paraffinic kerosene.

11. The process of claim 10, further comprising the step of formulating fuel with said synthetic paraffinic kerosene.

12. The process of claim 1, wherein said hyperthermophillic organism is selected from the group consisting of Pyrococcus, Thermococcus, Palaeococcus, Acidianus, Pyrobaculum, Pyrodictium, Pyrolobus, Methanopyrus, Methanothermus, Fervidobacterium and Thermotoga species, and combinations thereof.

13. The process of claim 1, wherein said culturing further produces acetate, and further comprising the step of using said acetate as a feedstock for an algal biomass.

14. The process of claim 1, wherein said culturing further produces carbon dioxide, and further comprising the step of using said carbon dioxide as a feedstock for an algal biomass.

15. The process of claim 1, wherein heat from culture of said biomass is exchanged with an algal biomass.

16. The process of claim 2, wherein said algal biomass is a residue after extraction of oil from said algal biomass.

17. The process of claim 1, wherein said culturing is at 80° C. or higher.

18. The process of claim 1, wherein said culturing is at about 80° C. to 110° C.

19. Straight chain paraffins produced by the process of claim 6.

20. Synthetic parafinnic kerosene produced by the process of claim 10.

21. Fuel, produced by the process of claim 11.
